(19) United States
(12) Patent Application Publication
(10) Pub. No.: US 2005/0049614 A1
(43) Pub. Date: Mar. 3, 2005

(54) Methods and instrumentation to treat obesity

(76) Inventor: Juan C. Cendan, Gainesville, FL (US)

Correspondence Address: Van Dyke & Associates, P.A., Suite 252, 7200 Lake Ellenor Drive, Orlando, FL, 32809, US

(21) Appl. No.: 10/634,285

(22) Filed: Aug. 5, 2003

(51) Int. Cl. A61B 17/08

(52) U.S. Cl. 606/139

(57) ABSTRACT

Disclosed herein is a novel surgical instrument particularly adapted for use in surgeries involving a circular anastomosis stapler, and methods of using same. The subject instrument allows for the easy manipulation of an anvil of a circular stapler during surgery, to allow for improved placement of the anvil. The subject instrument and methods provide an improved gastric bypass technique which alleviates the need to pass the anvil down the esophagus, or through an incision in the upper stomach portion.

### FIELD OF THE INVENTION

The present invention relates to the field of gastrointestinal surgery methods and instruments for the treatment of obesity.

### BACKGROUND OF THE INVENTION

Morbid obesity affects from about 3% to 5% of the population. The severely obese are at significantly greater risk of premature death, heart disease, stroke, diabetes mellitus, cancer, pulmonary diseases, orthopedic complications and accidents. The obese are also subject to discrimination in society, the workplace, etc. Several methods for treatment of morbid obesity include diets, pills, and other weight-reducing plans. Mechanical devices for insertion into the stomach, e.g., gastric balloons, to at least partially occupy the stomach have also been utilized. These approaches, however, are generally effective for a limited period of time. In addition, over 95% of those participating in such approaches regain their original weight, and, in many instances, gain additional weight.

Methods for treating obesity proven effective over the long term include surgery to restrict the amount of food consumed at one sitting and to change the digestive process such that less of the food consumed will be absorbed into the body. These procedures are collectively known as Bariatric Surgery and include Gastroplasty, Gastric Banding and Gastric Bypass.

Gastroplasty incorporates separating the stomach into two pouch areas, e.g., an upper pouch and a lower pouch, through stapling. A small opening or stoma is then formed through the row of staples. Thus, the consumed food collects within the upper pouch and passes through the stoma and into the lower pouch at a reduced rate thereby giving a sensation of fullness to the individual to limit the amount of food intake. Disadvantages of this procedure include expansion of the upper pouch and the stoma which thereby minimizes long term effectiveness of this procedure.

In Vertical Banded Gastroplasty (VBG), an upper gastric pouch is formed within the stomach by applying a vertical row of staples. A band (e.g., a Marlex mesh) is applied about the stomach adjacent the staple line to prevent dilation of the outlet port extending from the upper pouch into the remaining portion of the stomach. The Vertical Banded Gastroplasty (VBG) method, however, is subject to certain disadvantages including problematic post-operative healing, high rate of complications such as wound infection, pulmonary emboli, gastric perforation, gall bladder stones, etc.

Gastric Bypass combines the elements of intestinal rearrangement with a smaller stomach pouch. More particularly, with this procedure, the stomach is divided into an upper pouch and a lower pouch. The upper pouch, which receives the consumed food, is greatly reduced in capacity and is directly connected to the small intestine. However, conventional gastric bypass techniques involve invasive surgical approaches which have a deleterious effect on patient recovery and down time.

During a typical Gastric bypass procedure, the bowel is resected at a location below the duodenum. The end of the resected bowel is then conjoined with the upper pouch previously formed in the stomach. The bowel and stomach are joined together using circular anastomosis stapler. Such staplers have a removal anvil that must pass through the stomach and bowel thereby forming a circular hole (or anastomosis) for food to pass through. As this hole is formed, the stapler staples together the bowel and upper pouch. There are two recognized approaches for positioning the anvil so that the anastomosis may be formed. First, the anvil may be passed down the esophagus. This approach involves a number of risks, but primarily bears the risk puncturing the esophagus with the sharp end of the anvil, or lodging the anvil in the esophagus. Second, the anvil may be inserted into the upper pouch with an incision on the side of the upper pouch. While this approach avoids the risk of damaging the esophagus, it also has drawbacks. It creates an incision that must be sewed back up, and potentially narrow the pouch. And it is difficult for the surgeon to position the anvil at the most ideal situs in the upper pouch. Accordingly, there is a need for improved laparoscopic procedures for performing a gastric bypass procedure.

### SUMMARY OF THE INVENTION

Accordingly, the present disclosure is directed to surgical instrumentation and methods for performing a bypass procedure in a digestive system, which incorporates laparoscopic techniques to minimize surgical trauma to the patient. In one preferred embodiment, the subject invention is directed to an apparatus to facilitate the intracorporeal manipulation of a circular anastomotic stapler anvil. This embodiment allows for the safe handling of the anvil by utilizing a click-together construct. The tip of the instrument preferably comprises the ability to be deflected, or in other words is a “roticulating” end. Further, the apparatus is preferably a small size so that it may fit through standard laparoscopic ports. The size of the apparatus will allow for facile passage through a small enterotomy. Once through the enterotomy, the anvil can be attached to the end of the apparatus, thereby allowing safe and reproducible passage to the site of need.

According to another embodiment, the subject invention relates to a method of utilizing the subject apparatus in a modified roux-en-y gastric bypass procedure. According to this method, a lateral teat is made in the gastric pouch using conventional endoscopic staplers. A small enterotomy is made at the desired location proximate to the end of the staple line. A small portion of the teat is then excised. The subject apparatus described above is then passed through the enterotomy and out through the defect in the teat. The anvil of a conventional surgical stapler is then attached to the end of the apparatus. The apparatus with the anvil attached is directed back out of the enterotomy until the anvil is positioned at its desired location. The apparatus then releases the anvil, wherein the anvil is then mated with circular stapler. Upon firing the circular stapler, the circular gastrojejunostomy anastomosis is completed by conventional techniques. In addition, either before or after forming the gastrojejunostomy anastomosis, the lateral defect is easily closed with a standard linear stapler completing the usual linear proximal pouch; using this construct, the pouch will not be narrowed.

A method for performing a bypass procedure in a digestive system is also disclosed, which includes the steps of isolating an upper stomach portion of the stomach of a patient, resecting the bowel to define a bowel portion disconnected from the stomach, and connecting the bowel portion and the upper stomach portion. The step of connecting is preferably performed with a circular anastomosis instrument. The method may further include the step of introducing an anvil adapted for use with the anastomosis instrument through a side portion of the upper stomach portion and introducing a circular anastomosis instrument into the bowel portion. The anvil and the circular anastomosis instrument are connected, and the circular anastomosis instrument is fired to connect the bowel portion and the upper stomach portion.

The step of isolating may include positioning a linear stapler instrument about the stomach and firing the linear stapler to isolate the upper stomach portion with respect to the remainder of the stomach. The step of isolating preferably comprises the formation of a teat portion on the upper stomach portion. Similarly, the step of resecting includes positioning a linear stapler about the small bowel and firing the linear stapler. The linear stapler may have a knife blade associated therewith and wherein upon firing the knife blade is actuated to resect the bowel to define the bowel portion.

It is to be understood that the foregoing general description and the following detailed description are exemplary and explanatory only and are not to be viewed as being restrictive of the present, as claimed. These and other objects, features and advantages of the present invention will become apparent after a review of the following detailed description of the disclosed embodiments and the appended claims.

### BRIEF DESCRIPTION OF THE DRAWINGS

FIG. 1 shows a perspective view of an embodiment of the subject instrument.

FIG. 2 shows current art circular stapler anvil portions.

FIG. 3 shows one embodiment of a gastric bypass procedure according to the subject invention. FIG. 3A-F depict different steps of the procedure.

FIG. 4 shows a side view of one embodiment of the subject invention. FIG. 4A shows the mating portion of the embodiment designed for Ethicon-type Anvils. FIG. 4B shows the mating portion of the embodiment designed for Tyco-type anvils, which utilizes a coupler.

### DETAILED DESCRIPTION OF THE PREFERRED EMBODIMENTS

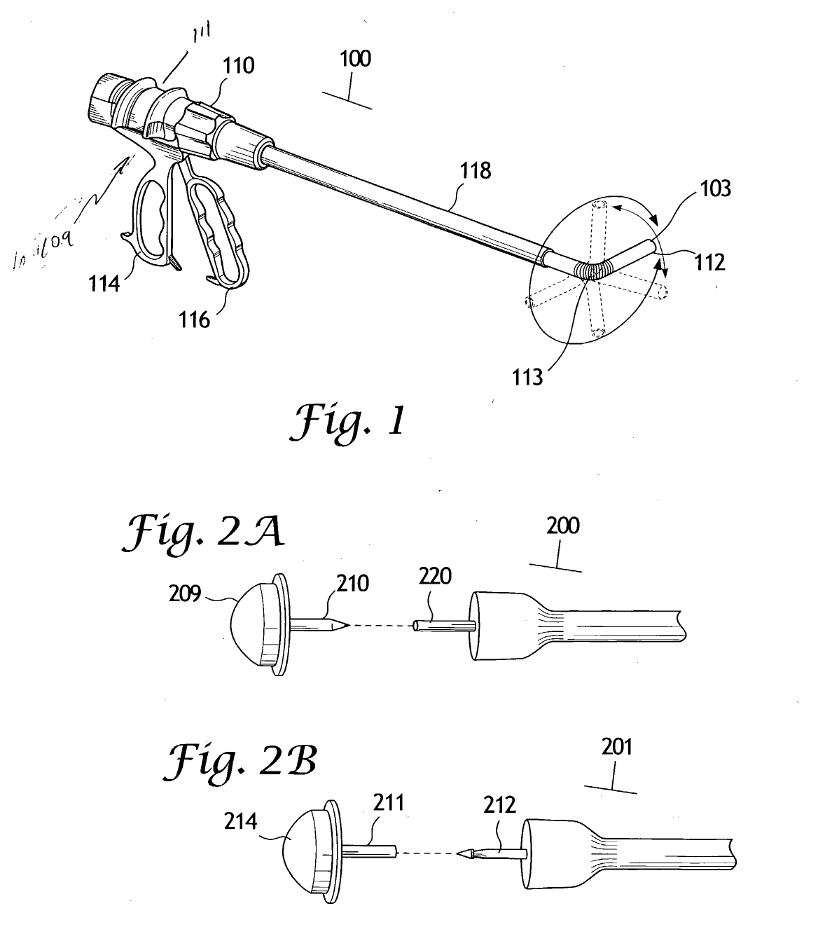

Turning to FIG. 1, shown is an exemplary embodiment 100 of the subject surgical instrument for use in manipulating various surgical components, e.g., anvil portions of conventional circular staplers. Instrument 100 comprises a elongated body portion 118. Extending from the elongated body portion is a flexible body portion 112, which can be deflected at a specified angle. In a preferred embodiment, the flexible body portion 112 comprises a hinge 113 at which the flexible body portion 112 may be bent (or deflected). Preferably, the flexible body portion 112 comprises a cross-sectional diameter of 3-15 mm. Preferably, the cross-sectional diameter is about 5 mm. At the end of the flexible portion 112, there is a tip 103 especially adapted for connecting or grasping the surgical component to be manipulated. At the proximal end of the instrument 100, there is a handle assembly 109. The handle assembly 109 comprises a handle grip 114 and a pivotally mounted actuating lever 116. When the actuating lever 116 is pivoted it controls the release of the surgical component inserted into tip 103. The instrument 100 comprises a rotatable adjusting knob 110 mounted at the distal end of the handle assembly 109 for rotating the body 118 and flexible body portion 112. The handle assembly also comprises a slidably mounted actuator 111 for controlling the articulating movement of the flexible body portion 112. As used herein, the term “reticulate” refers to the flexible body portion's 112 ability to be rotated and articulated for easy manipulation in tight spaces typically faced during laparoscopic techniques. The rotation may be in conjunction with rotation of the body 118 or separate.

Not to be construed as limiting, the internal mechanical components of the instrument could be constructed according to the instrument described in U.S. Pat. Nos. 5,829,662; and 6,464,711. Those skilled in the art equipped with the teachings herein would be readily able to build the necessary mechanical design of the instrument to achieve the rotating and articulating characteristics of the flexible body portion 112. Further, using skills and known materials, in view of the teachings herein, the skilled artisan would be able to build the releasing mechanism positioned in the tip 103 for releasing the surgical component. In a preferred embodiment, the surgical component to be grasped, manipulated and released is an anvil of a circular anastomosis stapler.

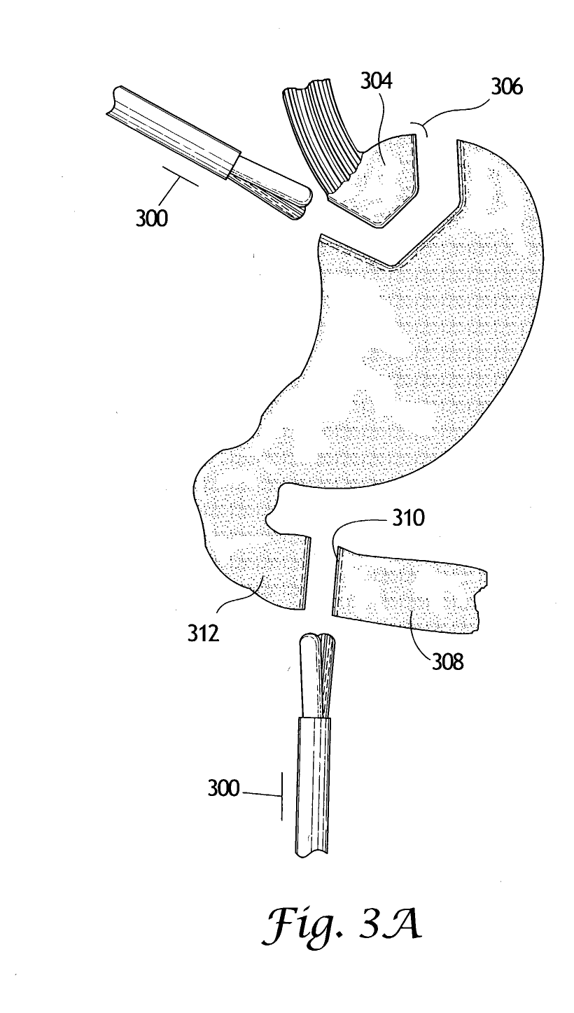

Shown in FIGS. 2A and B are examples of conventional circular stapler end portions and anvil portion. FIG. 2A shows an embodiment 200 illustrative of a type like the TYCO brand circular stapler and FIG. 2B shows an embodiment 201 illustrative of a type like the ETHICON brand circular stapler. See, for example, U.S. Pat. Nos. 5,327,914; 5,718,360; 4,603,693; and 5,104,025, for example. Embodiment 200 utilizes an anvil 209 comprising a pointed engagement end 210 that engages a receiver 220. When the anvil portions are engaged and the circular stapler fires, it produces a circular anastomosis at the intended site. Embodiment 201 comprises and anvil 214 comprising an engagement end 211 which acts a receiver for the pointed male end 212 of the circular stapler 201.

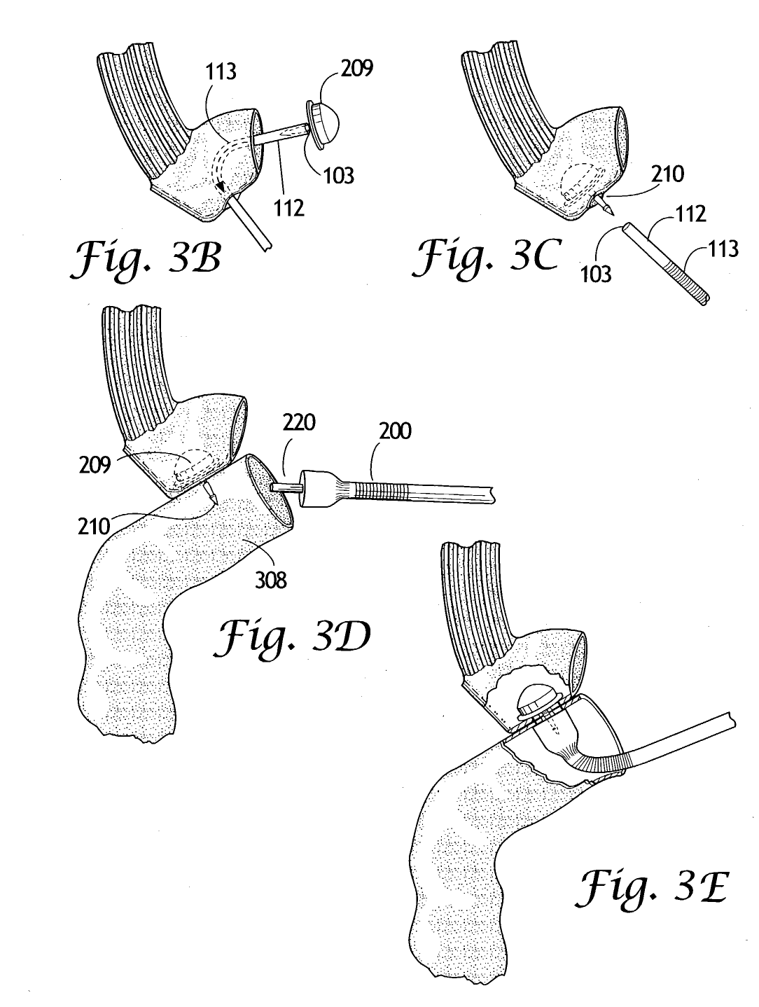

Shown in FIGS. 3A-F is a frame-by-frame depiction of a gastric bypass method utilizing the instrument 100 depicted in FIG. 1. According to this method, laparoscopic techniques preferably are utilized as conventionally performed in the art. Examples of such techniques are described at http://www.sabariatric.com/laparoscopic gbp.htm and U.S. Pat. No. 6,543,456 Typically, five small openings (each less than one-half inch long) are made in the abdomen. These openings allow the surgeon to pass a light, camera, and surgical instruments into the abdomen. The abdomen is inflated with gas (carbon dioxide) in order to allow the surgeon to get a better view of your stomach and internal structures. Once proper access to the gastrointestinal organs is achieved, the subject method involves the formation of an upper stomach pouch 304 using a conventional linear stapler 300, such as Endopath ETS Endoscopic Linear Cutter, made by Ethicon EndoSurgery, in Cincinnati, Ohio. Furthermore, the small intestine, preferably between the first 30-45 cm of the jejunum (even more preferred, distal to the Ligament of Treitz), 308 is transected with a linear stapler 300 at a transection line 310. An opening is made in the end of the transected intestine to insert a circular stapler as shown in FIGS. 3D-E. Also, it should be noted that the upper stomach pouch 304 is opened to pass the instrument 100 through its lower end and around to the side where the anvil 209 is inserted. According to conventional enteroenterostomy surgical techniques, the upstream portion of the small intestine 312 is attached downstream of the transection line 310. This allows drainage of pancreatic, biliary and gastric secretions to be delivered to the small intestine. The upper stomach portion 304 is cut to produce a protrusion or “teat” 306 at the side of the upper stomach portion 304. Turning to FIG. 3B, the flexible body portion 112 of the embodiment 100 is inserted at a location at the bottom of the upper stomach portion 304, and proximate to the suture line of the upper stomach portion 304, through a small opening created by the surgeon. The flexible body portion is deflected such that it may easily be passed through the upper stomach portion 304 and out of the teat 306. As mentioned above, the teat 306 is opened so that the anvil 209 may be grasped onto by the instrument 100. The anvil 209 is attached to the flexible body portion 112, at the tip 103.

The flexible body portion 112 is then pulled back out of the upper stomach portion 304 such that the anvil 209 is positioned into the desired location. The anvil 209 is then detached from the flexible body portion 112. See FIG. 3C. The transected small intestine 308 is abutted against the bottom of the upper stomach portion such that the engaging end of the anvil 210 passes through the transected small intestine 308. The receiving end of a conventional circular stapler 220 is then inserted into the end of the transected small intestine 308 (as mentioned supra, the transected end has been opened to allow passage of the circular stapler) and engaged to the anvil 209. The circular stapler is fired thereby creating an anastomosis (see FIG. 3D). The ends of the transected small intestine 308 and the teat 306 are then resected and sealed with a conventional linear stapler 300.

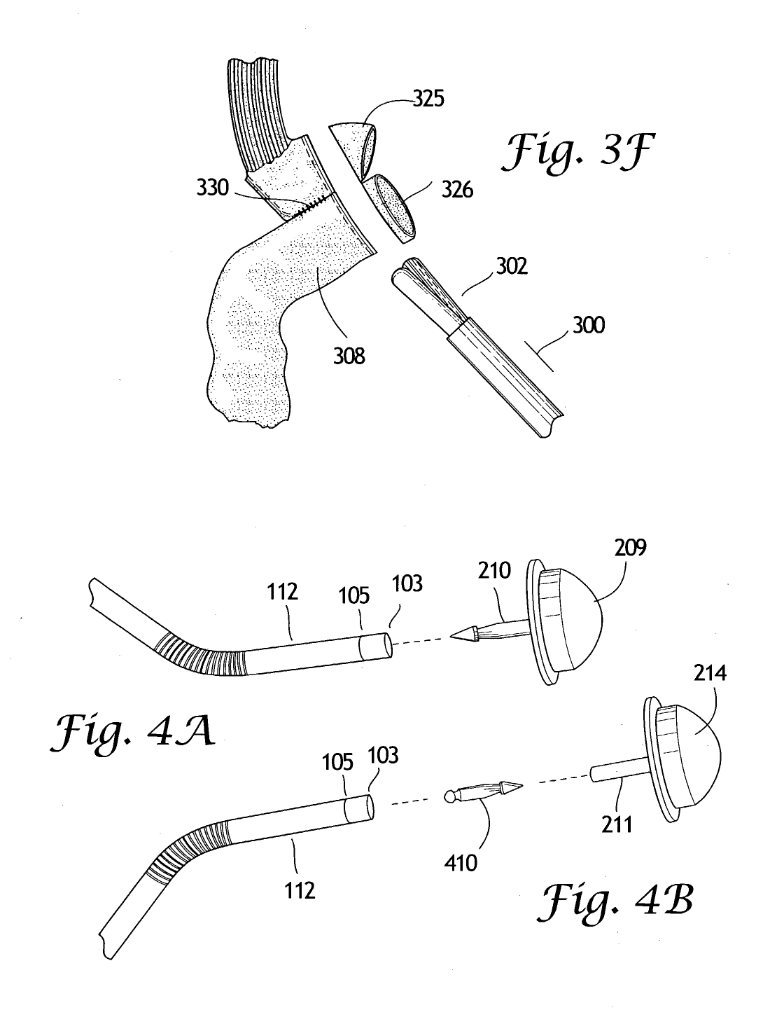

FIG. 4 shows the flexible body portion 112 and tip 103, and locking means 105 configured to engage the engagment end 210 of the Tyco-type anvil 209. In FIG. 4B, the subject invention provides a novel coupler 410 to assist in the engagement of engagement end 211 of the Ethicon-type anvil to the flexible body portion 112. Those skilled in the art will appreciate that any suitable locking means may be implemented into tip 103 of the flexible body portion 112. The locking means is preferably configured such that the engagement ends spring lock into place when inserted into the tip 103. A release mechanism is preferably interconnected to the locking means such that the release is actuated near the proximate end of the subject surgical instrument, preferably the pivotally mounted actuation lever 118.

It will be recognized that equivalent methods may be substituted for the methods illustrated and described herein and that the described embodiment of the invention is not the only method that may be employed to implement the claimed invention. Physicians may prefer to employ other types of surgery on the digestive system using an embodiment of the described surgical instrument. An embodiment described is in a Roux-en-y gastric bypass procedure. Other types of gastric bypass procedures may include those in which the stomach is not divided by severing a portion of it. A portion of the stomach is instead separated from the rest by staple lines only, as in a gastroplasty. Other type of gastric bypass procedures may further include those in which the gastric pouch is formed from alternate portions of the stomach such as a portion along the lesser curvature, rather than along the fundas as previously described. What is critical is that the anvil may be inserted into the GI tract without the need to pass it down the esophagus or by creating a large incision proximal to where the anvil needs to be positioned.

All patents, patent applications, publications, texts and references discussed or cited herein are incorporated by reference to the same extent as if each individual publication or patent application was specifically and individually set forth in its entirety. Nothing herein is to be construed as an admission that the invention is not entitled to antedate such disclosures by virtue of prior invention. In addition, all terms not specifically defined are first taken to have the meaning given through usage in this disclosure, and if no such meaning is inferable, their normal meaning. Where a limitation is described but not given a specific term, a term corresponding to such limitation may be taken from any references, patents, applications, and other documents cited herein, or, for an application claiming priority to this application, additionally from an Invention Disclosure Statement, Examiner's Summary of Cited References, or a paper otherwise entered into the file history of this application.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. Thus, for the above variations and in other regards, it should be understood that the examples and embodiments described herein are for illustrative purposes only and that various modifications or changes in light thereof will be suggested to persons skilled in the art and are to be included within the spirit and purview of this application and the scope of the appended claims.

1. A method for performing a bypass procedure in a digestive system comprising
isolating an upper stomach portion of the stomach of a patient;
introducing an anvil adapted for use with the anastomosis instrument through a side region of the upper stomach portion;
resecting the bowel to define a resected bowel portion; and
connecting the resected bowel portion and the upper stomach portion with a circular anastomosis instrument.

2. The method of claim 1, wherein isolating an upper stomach portion is conducted by utilizing a linear stapler, and wherein the upper stomach portion is cut such that a protrusion is formed on a side region of said upper stomach portion.

3. The method of claim 1, wherein introducing and anvil comprises the insertion of a surgical instrument through an opening in a bottom region of said upper stomach portion, wherein said flexible body portion has a tip adapted to releasably attach said anvil, and wherein said flexible body portion is capable of being reticulated.

4. The method of claim 3, wherein said anvil is attached to said tip and then carried through the upper stomach portion and positioned to rest in said opening.

5. The method of claim 4, wherein said connecting step comprises juxtaposing said bowel portion to said bottom region of said upper stomach portion by actuating a circular anastomosis stapler instrument, wherein said anvil is passed through said bottom region of said upper stomach portion and said bowel portion during said actuating of said circular anastomosis stapler instrument, thereby creating an anastamosis.

6. The method of claim 4 wherein said protrusion on said side region of said upper stomach portion is excised and sealed after said anvil is carried through said upper stomach portion.

7. A surgical instrument designed for manipulation of a surgical component comprising a cylindrical body portion comprising a proximal end and a distal end; a flexible body portion extending from distal end, said flexible body portion being designed to rotate and articulate and said flexible body portion comprising a tip adapted for releasably attaching said surgical component; and a handle assembly attached to or integral with said proximal end.

8. The surgical instrument of claim 7, wherein said handle assembly comprises an actuator whereby the moving of said actuator controls the articulating movement of said flexible body portion.

9. The surgical instrument of claim 7, wherein said handle assembly comprises an adjusting knob that controls the rotating movement of the flexible body portion.

10. The surgical instrument of claim 7, wherein said handle assembly comprises a pivotal handle lever that controls the release of said surgical component.

11. The surgical instrument of claim 7, wherein said surgical component is an anvil of a circular anastomosis stapler.
